(19) United States
(12) Patent Application Publication
(10) Pub. No.: US 2014/0275265 A1
(43) Pub. Date: Sep. 18, 2014

(54) THERAPEUTIC CREAM FOR APPLICATION TO SKIN

(71) Applicant: CORE PRODUCTS INTERNATIONAL, INC., Osceola, WI (US)

(72) Inventors: Philip H. Mattison, Forest Lake, MN (US); Paul Raymond Norstrem, Dresser, WI (US); Robert J. Bard, Northville, MI (US); Michael Artie Warshaw, Savannah, GA (US)

(73) Assignee: CORE PRODUCTS INTERNATIONAL, INC., Osceola, WI (US)

(21) Appl. No.: 14/202,512

(22) Filed: Mar. 10, 2014

Related U.S. Application Data
(60) Provisional application No. 61/777,117, filed on Mar. 12, 2013.

(51) Int. Cl. A61K 9/00; A61K 47/10; A61K 47/44; A61K 31/192

(52) U.S. Cl. CPC A61K 9/0014; A61K 31/192; A61K 47/10; A61K 47/44
USPC 514/570

(57) ABSTRACT

A stable, efficacious therapeutic cream comprising a non-steroidal anti-inflammatory drug (NSAID) is disclosed.

### CROSS-REFERENCE TO RELATED APPLICATIONS

This application claims the benefit of priority to U.S. Provisional Application No. 61/777,117, filed Mar. 12, 2013, which is hereby expressly incorporated by reference in its entirety.

### FIELD OF THE INVENTION

Topical therapeutic preparations and methods for treating inflammatory diseases and for reducing pain are disclosed. In certain embodiments, creams containing non-steroidal anti-inflammatory drugs (NSAIDs), for example, ibuprofen, are disclosed.

### BACKGROUND

The following includes information that may be useful in understanding the present embodiments. It is not an admission that any of the information provided herein is prior art, or relevant, to the presently described or claimed embodiments, or that any publication or document that is specifically or implicitly referenced is a prior art reference.

Topical routes of drug administration are often desirable, due to problems or limitations inherent in associated with other modes of drug delivery. For example, oral administration of drugs may be associated with variable absorption and metabolism of the drug and gastrointestinal irritation. In particular, the gastrointestinal irritation associated with the oral administration of non-steroidal anti-inflammatory drugs (NSAIDs), such as for example, aspirin, ibuprofen, and naproxen. (Lanza et al. “An endoscopic evaluation of the effects of non-steroidal anti-inflammatory drugs on the gastric mucosa,” Gastrointestinal Endoscopy, Volume 21, Issue 3, February 1975, Pages 103-105). Oral NSAID administration may cause irritation of the gastrointestinal tract (Davies “Toxicity of nonsteroidal anti-inflammatory drugs in the large intestine,” Diseases of the Colon & Rectum, Volume 38, Number 12, 1995, Pages 1311-1321). Oral NSAID administration may also result in other adverse events, such as headache, dizziness, salt and fluid retention, and hypertension.

In an effort to avoid the limitations of oral administration of NSAIDs, topical formulations have been developed such formulations may be applied directly to skin at or near the inflammation. Although such topical formulations may avoid some of the traditional limitations of administration of NSAIDs (e.g., gastrointestinal irritation), there exist known limitations of topical administration of NSAIDs. For example, the efficacy of topically applied drugs is often limited by poor skin penetration. (Reddy et al. “In Vitro Release of Ibuprofen from Different Topical Vehicles,” Asian Journal of Pharmaceutical Sciences and Research, Volume 1, Number 2, 2011, Pages 61-72).

The skin provides a protective barrier against foreign materials and infections and helps protect the body from water loss. The layer of skin primarily responsible for this barrier function is the epidermis, and more particularly, the uppermost layer of the epidermis, also referred to as the stratum corneum. The stratum corneum may provide a strong barrier to the diffusion of molecules through the skin, often restricting diffusion through the skin of molecules having a certain size, solubility, and/or lipophilicity. Thus, certain NSAIDs, such as ibuprofen, are difficult to administer topically because, under certain conditions, these NSAIDs have a relatively low solubility in typical carriers, and may precipitate and form an insoluble salts. Indeed, CA 2 500 907 to Schwarz et al. (“Schwarz”) highlights this very problem. Schwarz indicates that “[a] further method to increase drug solubility in the oil phase is to use highly polar compounds, miscible with the oil phase.” Schwarz at p. 4. Schwarz describes the use of ethoxyethylene glycol (Transcutol™) as one such highly polar compound. Id. Schwarz, however, teaches against the success of such a formulation because “upon mixing with water, most of the solvent is extracted into the water and the dissolved drug precipitates immediately and almost entirely from the oil phase.” Id.

Thus, there is a need for compositions containing NSAIDs useful for the relief of pain, inflammation, arthritis and/or associated symptoms that may be effectively delivered topically.

### DESCRIPTION OF THE RELATED TECHNOLOGY

U.S. Pat. No. 7,132,452 (prior publication: U.S. Publication No. 2004/0180066) discloses a topical formulation including a NSAID useful for alleviating pain/inflammation associated with infection caused by herpes virus, especially herpes simplex virus (HSV) and varicella-zoster virus (VZV). The area of administration of the topical formulation is generally near the mouth and lips. The topical formulations disclosed in U.S. Pat. No. 7,132,452 include greater than 10% by weight of alcohol components such as isopropyl alcohol.

U.S. Publication No. 2009/0123504 discloses a topical formulation including an NSAID and oleocanthal useful for reducing inflammation and/or pain associated with arthritis. The topical formulations disclosed in U.S. Publication No. 2009/0123504 generally include at least 50% by weight olive oil and do not include an aqueous component, but may optionally include water of up to about 10% (by weight of the composition). U.S. Publication No. 2009/0123504 discloses formulations that maintain the NSAID in its non-ionized form. This may be achieved by reducing, or entirely eliminating, aqueous components from the composition. U.S. Publication No. 2009/0123504 states that the NSAIDs in typical creams or oil-in-water emulsions are unable to effectively penetrate through the lipophilic external skin barrier, and an important fraction of the NSAID dose is lost.

Reddy et al. “In Vitro Release of Ibuprofen From Different Topical Vehicles,” Asian Journal of Pharmaceutical Sciences and Research, Volume 1, Number 2, 2011, Pages 61-72 discloses topical formulations including ibuprofen. Reddy et al. discloses a cream, gel or ointment as the topical formulation. An in-vitro diffusion cell experiment was designed to demonstrate the rate of release of ibuprofen from the three different topical vehicles: (i) an oil in water cream (ii) a gel and (iii) an ointment. In vitro release of ibuprofen from the three different bases to an aqueous receptor phase through cellophane membrane was monitored spectrophotometrically. Reddy et al. concludes the gel formulation could be a good candidate for the topical delivery of ibuprofen, giving higher drug release than the cream and ointment formulations.

### SUMMARY

The present disclosure provides aqueous therapeutic cream formulations for application to skin that allow effective penetration of NSAIDs through the external skin barrier.

Certain embodiments provide an aqueous cream for topical application to the skin comprising about 5% (w/w) to about 15% (w/w) diethylene glycol monoethyl ether, about 4% (w/w) to about 8% (w/w) tropical or vegetable oil, about 3% (w/w) to about 6.5% (w/w) ibuprofen, and about 56% (w/w) to about 68% (w/w) water, wherein said cream is stable at a temperature of 40° C. for a period of at least one month. In some embodiments, the aqueous cream comprises about 8% (w/w) to about 12% (w/w) diethylene glycol monoethyl ether, about 5% (w/w) to about 7% (w/w) tropical or vegetable oil, about 4% (w/w) to about 5.5% (w/w) ibuprofen, and about 60% (w/w) to about 64% (w/w) water, wherein said cream is stable at a temperature of 40° C. for a period of at least one month.

Certain embodiments provide an aqueous cream for topical application to the skin comprising greater than about 5% by weight diethylene glycol monoethyl ether, tropical or vegetable oil, an excipient component, ibuprofen, and water, wherein said cream is stable at temperatures of at least 40° C. for a period of at least one month. In some embodiments, the aqueous cream comprises greater than about 3% (w/w) of tropical or vegetable oil. In some embodiments, the aqueous cream comprises greater than about 3% (w/w) of ibuprofen. In some embodiments, the aqueous cream comprises greater than about 55% (w/w) of water. In some embodiments, the aqueous cream comprises greater than about 10% (w/w) of excipient component. In some embodiments, the aqueous cream comprises less than about 10% (w/w) of tropical or vegetable oil. In some embodiments, the aqueous cream comprises less than about 10% (w/w) of ibuprofen. In some embodiments, the aqueous cream comprises less than about 6% (w/w) of ibuprofen. In some embodiments, the aqueous cream comprises less than about 75% (w/w) of water. In some embodiments, the aqueous cream comprises less than about 25% (w/w) of excipient component. In some embodiments, the aqueous cream comprises about 0.2% (w/w) to about 12% (w/w), about 0.5% (w/w) to about 11% (w/w), about 1% (w/w) to about 10% (w/w), about 2% (w/w) to about 9% (w/w), about 4% (w/w) to about 8% (w/w), about 5% (w/w) to about 7% (w/w), or about 6% (w/w) of tropical or vegetable oil. In some embodiments, the aqueous cream comprises about 0.2% (w/w) to about 10.5% (w/w), about 0.6% (w/w) to about 9.5% (w/w), about 0.8% (w/w) to about 8.5% (w/w), about 1.5% (w/w) to about 7.5% (w/w), about 3% (w/w) to about 6.5% (w/w), about 4% (w/w) to about 5.5% (w/w), or about 5% (w/w) of ibuprofen. In some embodiments, the molar ratio of ibuprofen to coconut oil is in the range from about 10:1 to 1:10, about 5:1 to 1:5, about 2:1 to 1:2, or about 1:1.2. In some embodiments, the molar ratio of ibuprofen to diethylene glycol monoethyl ether is in the range from about 10:1 to 1:10, about 8:1 to 1:8, about 4:1 to 1:4, or about 1:2. In some embodiments, the molar ratio of water to ibuprofen is in the range from about 100:1 to 1:10, about 50:1 to 1:5, about 40:1 to 1:4, about 30:1 to 1:3, about 25:1 to 1:2, about 20:1 to 1:1, or about 12.5:1.

Certain embodiments provide an aqueous cream for topical application to the skin comprising tropical or vegetable oil, an excipient component, ibuprofen, water, and diethylene glycol monoethyl ether, wherein said cream is stable at a temperature of 40° C. for a period of at least one month.

Certain embodiments provide an aqueous cream for topical application to the skin comprising ibuprofen, tropical or vegetable oil, an excipient component, ibuprofen, water, and means for enhancing topical penetration, wherein said cream is stable at a temperature of 40° C. for a period of at least one month. In some embodiments, the tropical or vegetable oil is coconut oil.

Certain embodiments provide a therapeutic cream for application to skin comprising coconut oil, a trigylceride, glycerol, xanthan gum, PEG-6 stearate, ethylene glycol palmitostearate, PEG-32 stearate, diethylene glycol monoethyl ether, phenoxyethanol, propyl paraben, methyl paraben, ibuprofen, and water, wherein said cream is stable at a temperature of 40° C. for a period of at least one month.

Certain embodiments provide a method of reducing pain comprising applying to skin a composition as disclosed and described herein. In some embodiments, the pain is from inflammation. In some embodiments, the pain is of one or more joints. In some embodiments, the pain of one or more joints is from arthritis. In some embodiments, the method comprises greater than about 4% (w/w) of tropical or vegetable oil. In some embodiments, the method comprises greater than about 3% (w/w) of ibuprofen. In some embodiments, the method comprises greater than about 55% (w/w) of water. In some embodiments, the method comprises greater than about 10% (w/w) of excipient component. In some embodiments, the method comprises less than about 10% (w/w) of tropical or vegetable oil. In some embodiments, the method comprises less than about 10% (w/w) of ibuprofen. In some embodiments, the method comprises less than about 6% (w/w) of ibuprofen. In some embodiments, the method comprises less than about 70% (w/w) of water. In some embodiments, the method comprises less than about 20% (w/w) of excipient components. In some embodiments, the method comprises about 0.2% (w/w) to about 12% (w/w), about 0.5% (w/w) to about 11% (w/w), about 1% (w/w) to about 10% (w/w), about 2% (w/w) to about 9% (w/w), about 4% (w/w) to about 8% (w/w), about 5% (w/w) to about 7% (w/w), or about 6% (w/w) of tropical or vegetable oil. In some embodiments, the method comprises about 0.2% (w/w) to about 10.5% (w/w), about 0.6% (w/w) to about 9.5% (w/w), about 0.8% (w/w) to about 8.5% (w/w), about 1.5% (w/w) to about 7.5% (w/w), about 3% (w/w) to about 6.5% (w/w), about 4% (w/w) to about 5.5% (w/w), or about 5% (w/w) of ibuprofen. In some embodiments, the method comprises about 35% (w/w) to about 90% (w/w), about 40% (w/w) to about 85% (w/w), about 45% (w/w) to about 80% (w/w), about 50% (w/w) to about 75% (w/w), about 55% (w/w) to about 70% (w/w), about 60% (w/w) to about 65% (w/w), or about 62% (w/w) of water. In some embodiments, the method comprises about 5% (w/w) to about 20% (w/w), about 6% (w/w) to about 19% (w/w), about 7% (w/w) to about 18% (w/w), about 8% (w/w) to about 17% (w/w), about 9% (w/w) to about 16% (w/w), about 10% (w/w) to about 15% (w/w), or about 13% (w/w) of excipient component. In some embodiments, the molar ratio of ibuprofen to coconut oil is in the range from about 10:1 to 1:10, about 5:1 to 1:5, about 2:1 to 1:2, or about 1:1.2. In some embodiments, the molar ratio of ibuprofen to diethylene glycol monoethyl ether is in the range from about 10:1 to 1:10, about 8:1 to 1:8, about 4:1 to 1:4, or about 1:2. In some embodiments, the molar ratio of water to ibuprofen is in the range from about 100:1 to 1:10, about 50:1 to 1:5, about 40:1 to 1:4, about 30:1 to 1:3, about 20:1 to 1:2, about 15:1 to 1:1, or about 12.5:1.

### DETAILED DESCRIPTION

In the following detailed description, only certain exemplary embodiments have been shown and described, simply by way of illustration. As those skilled in the art would realize, the described embodiments may be modified in various different ways, all without departing from the spirit or scope of the present disclosure. Accordingly, the description is to be regarded as illustrative in nature and not restrictive. Since the disclosure may be modified in various ways and have various embodiments, the disclosure will be described in detail with reference to the examples. However, it should be understood that the disclosure is not limited to a specific embodiment but includes all changes and equivalent arrangements and substitutions included in the spirit and scope of the disclosure.

In accordance with the present disclosure, it has been discovered that non-steroidal anti-inflammatory drugs (NSAIDs) may be delivered topically for relief of inflammation and/or pain. More particularly, NSAIDs can be introduced into various compositions, such as creams, which may be topically applied at the source of inflammation and/or pain. Advantageously, the NSAID compositions overcome several of the drawbacks associated with traditional topical formulations.

NSAIDs are commonly used to treat inflammation and pain, and may be of particular use in treating the symptoms associated with arthritis. However, as noted above, oral administration of these medications may lead to gastrointestinal tract irritation. Furthermore, it has been difficult to administer ibuprofen and other similar NSAIDs such that they will permeate the stratum corneum. Consequently, formulating a NSAID-containing composition that is capable of penetrating through the stratum corneum in effective amounts is desired.

It has now been discovered that topical compositions comprising NSAIDs may be formulated such that the NSAID present in the composition will readily penetrate through skin. The compositions of the present disclosure are thus suitable for topical administration of NSAIDs to treat pain and/or inflammation, such as is commonly associated with arthritis and other conditions.

In certain embodiments, the composition may comprise an aqueous component. For example, the composition can be an aqueous cream for topical application. In some embodiments, the aqueous cream for topical application can comprise about 35% (w/w) to about 90% (w/w), about 40% (w/w) to about 85% (w/w), about 45% (w/w) to about 80% (w/w), about 50% (w/w) to about 75% (w/w), about 55% (w/w) to about 70% (w/w), about 60% (w/w) to about 65% (w/w), or about 62% (w/w) of water. In some embodiments, the aqueous cream for topical application can comprise at least 50% (w/w), 55% (w/w), 60% (w/w), 65% (w/w), 70% (w/w), 75% (w/w), 80% (w/w), or 85% (w/w) of water. In some embodiments, the aqueous cream for topical application can comprise up to 50% (w/w), 55% (w/w), 60% (w/w), 65% (w/w), 70% (w/w), 75% (w/w), 80% (w/w), or 85% (w/w) of water. In some embodiments, the aqueous cream for topical application can comprise 50% (w/w), 55% (w/w), 60% (w/w), 65% (w/w), 70% (w/w), 75% (w/w), 80% (w/w), or 85% (w/w) of water or a range defined by any two of the preceding values.

In certain embodiments, the compositions of the present disclosure may include any NSAID and can be used for treatment of inflammation and/or pain, such as is associated with different types of arthritic disorders, injuries, and other disorders.

In certain embodiments, the NSAID can be selected from the groups consisting of ibuprofen, aspirin, difunisal, etodolac, indometacin, nabumeton, sulindac, tolmetin, caprofen, fenbufen, flubiprofen, ketoprofen, ketorolac, loxoprofen, naproxen, oxaprozin, tiaprofenic acid, suprofen, mefenaminic acid, phenylbutazone, piroxicam, meloxicam, celecoxib, etoricoxib, lumiracoxib, parecoxib, rofecoxib, valdecoxib, and nimesulide, or combinations thereof. In a typical embodiment, the NSAID can be ibuprofen or a pharmaceutically acceptable salt thereof. In some embodiments, the composition contains a single active ingredient. In certain embodiments, the composition contains an NSAID as the sole active ingredient in the composition. In some embodiments, the composition contains ibuprofen as the only active ingredient in the composition.

The amount of NSAID present in a particular disclosed composition may vary. The amount of NSAID will depend on the ability of the individual NSAID to penetrate through the skin, the NSAIDs potency, and, for example, its half-life, but will preferably be present in an amount effective to reduce inflammation and/or pain. Therapeutically effective dosages of the NSAID can range from 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, from about 35 mg to about 40 mg per dose, from about 40 mg to about 50 mg per dose, from about 50 mg to about 75 mg per dose, from about 75 mg to about 100 mg per dose, from about 100 mg to about 200 mg per dose, from about 200 mg to about 400 mg per dose, from about 400 mg to about 600 mg per dose, from about 600 mg to about 800 mg per dose, or from about 800 mg to about 5000 mg per dose. An effective amount of the NSAID is ordinarily supplied at a dosage level of from about 0.01 mg/kg to about 150 mg/kg of body weight per day. Typically, the range is from about 0.1 to about 80 mg/kg of body weight per day, and especially from about 0.2 mg/kg to about 40 mg/kg of body weight per day. The NSAID may be administered on a regimen of about 1 to about 10 times per day for a period of from about 1, 5, 10, 15, 30 or 60 minutes, or 1, 2, 3, 5, 7, 10, 12, 15, 18, 20 or 24 hours or a range defined by any two of the preceding values.

Some embodiments provide an aqueous cream for topical application including skin penetration enhancers.

Examples of suitable skin penetration enhancers include alcohols, fatty acids, fatty acid esters, polyols, sulphoxides, glyceryl monooleate, lauryl lactate, Dodecyl-2-(N,N-dimethyl)-amino propionate (DDAIP), N-(4-bromobenzoyl)-S,S-dimethyliminosulfurane, NexACT enhancers, 2-nonyl-1,3-dioxolane (SEPA®), 1-dodecylazacycloheptan-2-one (Azone®), pyrrolidones, essential oil, terpenes, terpenoids, oxazolidinones, urea and the like.

Examples of suitable fatty acids include, but are not limited to, valeric acid, heptanoic acid, pelagonic acid, caproic acid, capric acid, lauric acid, myristic acid, stearic acid, oleic acid, and caprylic acid; and branched fatty acids, such as isovaleric acid, neopentanoic acid, neoheptanoic acid, neononanoic acid, trimethyl hexanoic acid, neodecanoic acid, and isostearic acid.

Examples of suitable fatty acid esters include but are not limited to, isopropyl n-butyrate, isopropyl n-hexanoate, isopropyl n-decanoate, isopropyl myristate, isopropyl palmitate, and octyldodecyl myristate; alkyl fatty acid esters such as ethyl acetate, butyl acetate, methyl acetate, methylvalerate, methylpropionate, diethyl sebacate, and ethyl oleate; and diisopropyl adipate and dimethyl isosorbide.

Suitable skin penetration enhancers are known in the art and include, but are not limited to, monoglycerides, polyglycosylated glycerides, glyceryl monoethyl ether, polysorbates, beta-cyclodextrin, cyclopentadecalactone, alkyl-2-(N,N-disubstituted amino)-alkanoate ester, 2-(n-nonyl)-1,3-dioxolane, isopropyl myristate, terpinol, menthol, cineol, monoolein, sodium oleate, oleyl oleate, laurylcapram, bisabolol, capaicin, and capsicum. Other examples of suitable skin penetration enhancers and a description of their mechanism of action may be found in Goodman and Barry, “Percutaneous Absorption,” in Mechanisms-Methodology-Drug Delivery, 2nd Edition, Bronaugh and Maibach, eds., 1989, pp. 567-593, Marcel Dekker, Inc., NY.

In certain embodiments, the skin penetration enhancer can be selected from the group consisting of n-octanol, D-limonene, oleic acid, cineol, isopropyl myristate, monooleate, monoolein, sodium oleate, oleyl oleate, laurylcapram, sodium lauryl sulfate, bisabolol, lauric acid, myristic acid, isopropyl palmitate, diisopropyl adipate, dimethyl isosorbide, propylene glycol, butylene glycol, polyethylene glycol, dipropylene glycol, ethoxydiglycol, and pentylene glycol or combinations thereof. In a typical embodiment, the skin penetration enhancer can be selected from the group consisting of oleic acid, laurocapram, sodium lauryl sulphate, bisabolol, lauric acid, myristic acid, isopropyl myristate, isopropyl palmitate, diisopropyl adipate, dimethyl isosorbide, propylene glycol, butylene glycol, polyethylene glycol, dipropylene glycol, ethoxydiglycol, and pentylene glycol, or combinations thereof.

In certain embodiments, the aqueous cream for topical application can comprise at least 5% (w/w), 6% (w/w), 7% (w/w), 8% (w/w), 9% (w/w), 10% (w/w), 11% (w/w), or 12% (w/w) of a skin penetration enhancer. In some embodiments, the aqueous cream for topical application can comprise 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of a skin penetration enhancer or a range defined by any two of the preceding values. In a typical embodiment, the skin penetration enhancer can be ethoxydiglycol.

In certain embodiments, the composition can include an emollient. In some embodiments, the emollient can be alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, glycerol esters and derivatives, propylene glycol esters and derivatives, alkoxylated carboxylic acids, alkoxylated alcohols, fatty alcohols, and combinations thereof. In some embodiments, the emollient can be selected from the group consisting of cetyl palmitate, stearyl palmitate, cetyl stearate, isopropyl laurate, isopropyl myristate, and isopropyl palmitate, or combinations thereof. In some embodiments, the emollient can be selected from the group consisting of octyldodecanol, lauryl, myristyl, cetyl, stearyl, and behenyl alcohol, or combinations thereof. In some embodiments, the emollient can be selected from the group consisting of eucalyptol, ceteraryl glucoside, dimethyl isosorbic polyglyceryl-3 cetyl ether, polyglyceryl-3 decyltetradecanol, propylene glycol and myristyl ether, or combinations thereof.

In certain embodiments, the aqueous cream for topical application can comprise at least 1% (w/w), 2% (w/w), 3% (w/w), 4% (w/w), 5% (w/w), 6% (w/w), 7% (w/w), 8% (w/w), 9% (w/w), 10% (w/w), 11% (w/w), or 12% (w/w) of an emollient. In some embodiments, the aqueous cream for topical application can comprise 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of an emollient or a range defined by any two of the preceding values.

In certain embodiments, the aqueous cream for topical application can comprise natural fats and oils. In some embodiments, the natural fats and oils can be selected from the group consisting of citrus oil, olive oil, avocado oil, apricot oil, babassu oil, borage oil, camellia oil, canola oil, castor oil, coconut oil, corn oil, cottonseed oil, emu oil, evening primrose oil, hydrogenated cottonseed oil, hydrogenated palm kernel oil, maleated soybean oil, meadowfoam oil, palm kernel oil, peanut oil, rapeseed oil, grapeseed oil, safflower oil, sphingolipids, seed almond oil, tall oil, lauric acid, palmitic acid, stearic acid, linoleic acid, stearyl alcohol, lauryl alcohol, myristyl alcohol, behenyl alcohol, rose hip oil, calendula oil, chamomile oil, eucalyptus oil, juniper oil, sandlewood oil, tea tree oil, sunflower oil, and soybean oil, or combinations thereof.

In certain embodiments, the aqueous cream for topical application can comprise at least 5% (w/w), 6% (w/w), 7% (w/w), 8% (w/w), 9% (w/w), 10% (w/w), 11% (w/w), or 12% (w/w) of a vegetable oil. In some embodiments, the aqueous cream for topical application can comprise 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of a vegetable oil or a range defined by any two of the preceding values. In a typical embodiment, the aqueous cream for topical application can comprise coconut oil.

In certain embodiments, the aqueous cream for topical application may optionally further comprise ingredients to relieve irritation, such as anti-itch agents. In some embodiments, the anti-itch agents may be present in the aqueous cream for topical application in an amount of from about 0.1% to about 33% (w/w), more typically, from about 0.5% to about 5% (w/w). Examples of suitable anti-itch agents are listed below, as well as the preferred concentration for each agent, given in percent by weight of the composition: lauromacrogols, benzocaine (about 5% to about 20%), butamben picrate (about 1%), dibucaine (about 0.25% to about 1%), dibucaine hydrochloric acid (0.25% to about 1%), dimethisoquin hydrochloric acid (about 0.3% to about 0.5%), dyclonine hydrochloric acid (about 0.5% to about 1%), lidocaine (about 0.5% to about 5%), lidocaine hydrochloric acid (about 0.5% to about 5%), pramoxine hydrochloric acid (about 0.5% to about 1%), tetracaine (about 1% to about 2%), tetracaine hydrochloric acid (about 1% to about 2%), benzyl alcohol (about 10% to about 33%), camphor (about 0.1% to about 3%), juniper tar (about 1% to about 5%), menthol (about 0.1% to about 1%), phenol (about 0.5% to about 1.5%), phenolate sodium (about 0.5% to about 1.5%), resorcinol (about 0.5% to about 3%), diphenhydramine hydrochloric acid (about 1% to about 2%), tripelennamine hydrochloric acid (about 0.5% to about 2%), hydrocortisone (about 0.1% to about 5%, preferably about 0.5% to about 2.5%), and combinations thereof. In some embodiments, the aqueous cream for topical application may optionally also comprise cosmetic anti-itch ingredients such as, for example, Symcalmin® (Symrise GmbH & Co., Holzminden, Germany).

In certain embodiments, the compositions may include adjunct components conventionally found in pharmaceutical compositions in their art-established fashion and at their art-established levels. For example, the compositions may comprise additional compatible pharmaceutically active materials for combination therapy, such as antimicrobials, antioxidants, anti-parasitic agents, antipruritics, antifungals, antiseptic actives, biological actives, astringents, keratolytic actives, local anaesthetics, anti-stinging agents, anti-reddening agents, skin soothing agents, and combinations thereof.

In certain embodiments, the compositions may include colorants, deodorants, fragrances, perfumes, emulsifiers, anti-foaming agents, lubricants, natural moisturizing agents, skin conditioning agents, skin protectants and skin benefit agents (e.g., aloe vera and laponite), solvents, solubilizing agents, suspending agents, wetting agents, humectants, preservatives, propellants, dyes and/or pigments, and combinations thereof. In some embodiments, the compositions may have a particularly pleasant fragrance. In some embodiments, the compositions may have a particularly pleasant texture. In some embodiments, the compositions may have a particularly pleasant soothing effect.

In certain embodiments, the compositions may include excipients conventionally found in topical compositions.

In certain embodiments, the excipients can include a viscosity-adjusting agent. In some embodiments, the viscosity adjusting agents can be selected from the group consisting of long chain alcohols, cellulose ethers, gums, magnesium aluminum silicate, silica, microcrystalline wax, beeswax, paraffin, and cetyl palmitate, homopolymers, and copolymers. In some embodiments, the long chain alcohols can be cetyl alcohol, stearyl alcohol, or cetearyl alcohol. In some embodiments, the cellulose ethers can be hydroxypropylmethylcellulose, hydroxyethylcellulose, hydroxypropylcellulose, or carboxymethylcellulose. In some embodiments, the gum can be xanthan gum or sclerotium gum. In a particular embodiment, the viscosity adjusting agents can include xanthan gum. In another embodiment, the viscosity adjusting agents is xanthan gum. In another embodiment, the viscosity adjusting agent is a polyalkylene oxide such as polyethylene glycol. In other embodiments, the viscosity adjusting agent is pullulan. In other embodiments, the viscosity adjusting agent is a polyvinyl halide, such as polyvinyl chloride.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a viscosity adjusting agent. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a viscosity adjusting agent or a range defined by any two of the preceding values.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of xanthan gum. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of xanthan gum or a range defined by any two of the preceding values.

In certain embodiments, the excipients can include a topical pharmaceutical and cosmetically-acceptable emollient. As used herein, “emollients” refer to materials used for the prevention or relief of dryness, as well as for the protection of the skin. Sagarin, Cosmetics, Science and Technology, 2nd Edition, Vol. 1, pp. 32-43 (1972), contains numerous examples of suitable materials for use as emollients. Examples of classes of useful emollients include, but are not limited to, hydrocarbon oils and waxes such as mineral oil, petrolatum, paraffin, ceresin, ozokerite, microcrystalline wax, polyethylene, and perhydrosqualene; silicone oil, such as dimethyl polysiloxanes, methylphenyl polysiloxanes, and water-soluble and alcohol-soluble silicone glycol copolymers. Other suitable emollients include triglyceride esters such as vegetable and animal fats and oils including castor oil, safflower oil, cotton seed oil, corn oil, olive oil, cod liver oil, almond oil, avocado oil, palm oil, sesame oil, and soybean oil; acetoglyceride esters, such as acetylated monoglycerides; ethoxylated glycerides, such as ethoxylated glycerylmonostearate; alkyl esters of fatty acids including methyl, isopropyl, and butyl esters of fatty acids, alkyl esters including hexyl laurate, isohexyl laurate, iso-hexyl palmitate, isopropyl palmitate, decyl oleate, isodecyl oleate, hexadecyl stearate, decyl stearate, isopropyl isostearate, diisopropyl adipate, dissohexyl adipate, di-hexyldecyl adipate, di-isopropyl sebacate, lauryl lactate, myristyl lactate, and cetyl lactate; and alkenyl esters of fatty acids such as oleyl myristate, oleyl stearate, and oleyl oleate. Other suitable classes of emollients include fatty acids such as pelargonic, lauric, myristic, palmitic, stearic, isostearic, hydroxystearic, oleic, linoleic, ricinoleic, arachidic, behenic, and erucic acids; fatty alcohols such as lauryl, myristyl, cetyl, hexadecyl, stearyl, isostearyl, hydroxystearyl, oleyl, ricinoleyl, behenyl, and erucyl alcohols, as well as 2-octyl dodecanol; fatty alcohol ethers; ethoxylated fatty alcohols; ether-esters such as fatty acid esters of ethoxylated fatty alcohols; lanolin and derivatives including lanolin oil, lanolin wax, lanolin alcohols, lanolin fatty acids, isopropyllanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysis of lanolin, ethoxylated hydrogenated lanolin, ethoxylated sorbitol lanolin, and liquid and semisolid lanolin absorption bases are illustrative of emollients derived from lanolin; polyhydric alcohols and polyether derivatives such as propylene glycol, dipropylene glycol, polypropylene glycols, polyoxyethylene polyoxypropylene glycols, polyoxypropylene polyoxyethylene glycols, glycerol, sorbitol, ethoxylated sorbitol, hydroxypropylsorbitol, polyethylene glycols, methoxy polyethylene glycols, polyalkylene glycols and derivatives, hexylene glycol(2-methyl-2,4-pentanediol), 1,3-butylene glycol, 1,2,6-hexanetriol, 2-ethyl,3-hexanediol, and polyoxypropylene derivatives of trimethylolpropane; polydydric alcohol esters such as ethylene glycol mono- and di-fatty acid esters, diethylene glycol mono- and di-fatty acid esters, polyethylene glycol, mono- and di-fatty acid esters, propylene glycol mono- and di-fatty esters, polypropylene glycol monooleate, polypropylene glycol monostearate, ethoxylatedpropylene glycol monostearate, glyceryl mono- and di-fatty acid esters, polyglycerol poly-fatty acid esters, ethoxylated glyceryl monostearate, 1,3-butylene glycolmonostearate, 1,3-butylene glycol distearate, polyoxyethylene polyol fatty acid ester, sorbitan fatty acid esters, and polyoxyethylene sorbitan fatty acid esters; wax esters such as beeswax, spermaceti, myristyl myristate and stearyl stearate; beeswax derivatives, e.g., polyoxyethylene sorbitol beeswax; vegetable waxes including carnauba and candelilla waxes; and phospholipids, such as lecithin and derivatives; sterols including, for example, cholesterol and cholesterol fatty acid esters; amides such as fatty acid amides, ethoxylated fatty acid amides and solid fatty acid alkanolamides. In some embodiments, the emollient can be selected from the group consisting of glycerol, hexanetriol, butanetriol, lactic acid, urea, pyrrolidone carboxylic acid, amino acids, guanidine, diglycerol and triglycerol. In a typical embodiment, the emollient can include glycerol.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of an emollient. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of an emollient or a range defined by any two of the preceding values.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of glycerol. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of glycerol or a range defined by any two of the preceding values.

In certain embodiments, the excipients can include fatty acid triglycerides, namely the triglycerol esters of saturated and/or unsaturated, branched and/or unbranched alkanecarboxylic acids having a chain length of from 6 to 24 carbon atoms, in particular 6-10 carbon atoms. In a particular embodiment, the ester oil can be caprylic/capric triglyceride.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid triglyceride. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid triglyceride or a range defined by any two of the preceding values.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of caprylic/capric triglyceride. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of caprylic/capric triglyceride or a range defined by any two of the preceding values.

In certain embodiments, the excipients can include fatty acid triglycerides, namely the triglycerol esters of saturated and/or unsaturated, branched and/or unbranched alkanecarboxylic acids having a chain length of from 6 to 24 carbon atoms, in particular 6-10 carbon atoms. In a particular embodiment, the fatty acid triglyceride can be caprylic/capric triglyceride.

In certain embodiments, the excipients can include fatty acid diglycerides, namely the diglycerol esters of saturated and/or unsaturated, branched and/or unbranched alkanecarboxylic acids having a chain length of from 6 to 24 carbon atoms, in particular 6-10 carbon atoms.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid diglyceride. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid diglyceride or a range defined by any two of the preceding values.

In certain embodiments, the excipients can include fatty acid monoglycerides, namely the monoglycerol esters of saturated and/or unsaturated, branched and/or unbranched alkanecarboxylic acids having a chain length of from 6 to 24 carbon atoms, in particular 6-10 carbon atoms.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid monoglyceride. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid monoglyceride or a range defined by any two of the preceding values.

In certain embodiments, the excipients can include an emulsifier. Suitable emulsifiers are disclosed in, for example, in McCutcheon's Detergents and Emulsifiers, North American Edition, pp. 317-324 (1986), and the ICI Handbook, pp. 1673-1686. In some embodiments, the emulsifier can include glycerol monostearate. In some embodiments, the emulsifier can include polyoxyl stearate. In some embodiments, the emulsifier can include glycerol monostearate and polyoxyl stearate. In some embodiments, the emulsifier can include PEG-6 Stearate and Glycol stearate and PEG-32 stearate. In some embodiments, the emulsifier can include glycerol monostearate, PEG-6 Stearate, Glycol stearate and PEG-32 stearate.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier or a range defined by any two of the preceding values. In some embodiments, the emulsifier can include one or more components, two or more components or three or more components.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate or a range defined by any two of the preceding values. In some embodiments, the can be a mixture of glycerol monostearate, PEG-6 Stearate, Glycol stearate and PEG-32 stearate.

In certain embodiments, the excipients can include preservatives. In some embodiments, the preservatives can be selected from the group consisting of benzyl alcohol, methyl paraben, propyl paraben, DMDM hydantoin, methylchloroisothiaoline, methylisothiazolinone, imidazolidinyl urea phenoxyethanol, sodium benzoate and benzoic acid. In some embodiments, the preservatives can include phenoxyethanol, propyl paraben, and methyl paraben. In some embodiments, the preservatives can include benzalkonium chloride and/or poly(hexamethylenebiguanide) hydrochloride (PHMB).

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative or a range defined by any two of the preceding values. In some embodiments, the preservative can include one or more components, two or more components or three or more components.

In certain embodiments, the aqueous cream for topical application can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative including phenoxyethanol, propyl paraben, and methyl paraben. In some embodiments, the aqueous cream for topical application can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative including phenoxyethanol, propyl paraben, and methyl paraben or a range defined by any two of the preceding values.

In certain embodiments, the compositions may include colorants, deodorants, fragrances, perfumes, anti-foaming agents, lubricants, natural moisturizing agents, skin conditioning agents, skin protectants, skin benefit agents, solvents, solubilizing agents, suspending agents, wetting agents, humectants, propellants, dyes, pigments, and combinations thereof.

In certain embodiments, the compositions may include additional components added to enhance the odor, texture or color of the composition. For example, fragrances may be added to enhance odor. For example, emulsifiers or inert spheres may be added to enhance texture. For example, colorants may be added to enhance color.

In certain embodiments, the compositions are useful in treating, and thus may be used to treat, inflammation and/or pain, such as is commonly associated with different types of arthritic disorders, injury related discomfort, and other such conditions. The composition may be applied to a body portion, such as a hand, foot, knee, elbow, and the like to treat pain and/or inflammation of the body portion. The composition may be applied by any suitable means, such as rubbing, spraying, rolling, wiping, and the like, and massaged into the body portion to be treated.

Certain embodiments provide aqueous therapeutic cream formulations that do not include components that may be considered to have certain negative properties. In some embodiments, the aqueous therapeutic cream formulations do not include stearic acid. In some embodiments, the aqueous therapeutic cream formulations do not include potassium hydroxide. In some embodiments, the aqueous therapeutic cream formulations do not include sodium hydroxide. In some embodiments, the aqueous therapeutic cream formulations do not include cetyl alcohol. In some embodiments, the aqueous therapeutic cream formulations do not include propylene glycol. In some embodiments, the aqueous therapeutic cream formulations do not include methyl p-hydroxybenzoate. In some embodiments, the aqueous therapeutic cream formulations do not include dimethyl sulfoxide. In some embodiments, the aqueous therapeutic cream formulations do not include methyl methanesulfonate. In some embodiments, the aqueous therapeutic cream formulations do not include methanol, ethanol, propanol, isopropanol, butanol, sec-butanol, tert-butanol, pentanol, hexanol, amyl alcohol, Cetyl alcohol, ethylene glycol, erythritol, xylitol, mannitol, sorbitol, menthol. In some embodiments, the aqueous therapeutic cream formulations do not include ethanol. In some embodiments, the aqueous therapeutic cream formulations do not include propanol or isopropanol. In some embodiments, the aqueous therapeutic cream formulations do not include carboxypolymethylene (Carbopol™). In some embodiments, the aqueous therapeutic cream formulations do not include lauric acid. In some embodiments, the aqueous therapeutic cream formulations do not include vitamin E. In some embodiments, the aqueous therapeutic cream formulations do not include oleocanthal. In some embodiments, the aqueous therapeutic cream formulations do not include olive oil. In some embodiments, the aqueous therapeutic cream formulations do not include beeswax. In some embodiments, the aqueous therapeutic cream formulations do not include potassium bicarbonate. In some embodiments, the aqueous therapeutic cream formulations do not include Labrasol® or Labrafil®.

### EXAMPLES

The following examples are meant to further illustrate the embodiments, they are not meant to be limiting in any way.

### Example 1

In this example, a water, Diethylene Glycol Monoethyl Ether (Transcutol™), coconut oil and ibuprofen-containing topical therapeutic formulation was produced. The following components were used in the preparation.

| Batch | | Percentage | Batch Size | Amount |
|---|---|---|---|---|
| No. 1 | Ingredients | (%) | (g) | (g) |
| | | | 500 | |
| 1 | Water | 62.27 | | 311.35 |
| 2 | Xanthan Gum | 1.03 | | 5.15 |
| 3 | Glycerol | 2.1 | | 10.50 |
| | (Glycerin) | | | |
| 4 | Caprylic/Capric | 1.0 | | 5.00 |
| | Triglyceride | | | |
| 5 | Coconut Oil | 6.0 | | 30.00 |
| 6 | Polyoxyl | 11.6 | | 58.00 |
| | stearate, Glycol | | | |
| | stearate | | | |
| | (Tefose 63) | | | |
| 7 | Ibuprofen | 5.0 | | 25.00 |
| 8 | Diethylene | 10.0 | | 50.00 |
| | Glycol | | | |
| | Monoethyl | | | |
| | Ether | | | |
| | (Transcutol ™) | | | |
| 9 | Phenoxyethanol, | 1.0 | | 5.00 |
| | Propyl Paraben, | | | |
| | Methyl Paraben | | | |
| Total | | 100 | | |

The formulation was prepared as follows:

1) Diethylene glycol monoethyl ether (Transcutol™), methyl paraben and water were combined in a mixing vessel and heated to 65° C.

2) Polyoxyl stearate, glycol stearate (Tefose 63); coconut oil; propyl paraben; caprylic/capric triglyceride; and ibuprofen were combined in a second mixing vessel and heated to 65° C.

3) The above described mixtures were then combined and allowed to cool to 63° C. Xanthan gum and glycerin were then added.

4) When the temperature of the mixture reaches about 45° C. to about 50° C., phenoxyethanol was combined to afford the therapeutic formulation.

A sample of the above-described therapeutic formulation had a pH of between 4.0 to 6.0 and a viscosity of 9,000 cps to 11,000 cps.

### Example 2

In this example, a water, Diethylene Glycol Monoethyl Ether (Transcutol™), coconut oil and ibuprofen-containing topical therapeutic preparation was produced. The following components were used in the preparation.

| Batch | | Percentage | Batch Size | Amount |
|---|---|---|---|---|
| No. 2 | Ingredients | (%) | (g) | (g) |
| | | | 500 | |
| 1 | Water | 68.14 | | 340.7 |
| 2 | Xanthan Gum | 0.36 | | 1.8 |
| 3 | Glycerol | 2.0 | | 10 |
| | (Glycerin) | | | |
| 4 | Caprylic/Capric | 2.0 | | 10 |
| | Triglyceride | | | |
| 5 | Coconut Oil | 4.5 | | 22.5 |
| 6 | Polyoxyl | 8.0 | | 40 |
| | stearate, Glycol | | | |
| | stearate | | | |
| | (Tefose 63) | | | |
| 7 | Ibuprofen | 5.0 | | 25.00 |
| 8 | Diethylene | 10.0 | | 50.00 |
| | Glycol | | | |
| | Monoethyl | | | |
| | Ether | | | |
| | (Transcutol ™) | | | |
| 9 | Phenoxyethanol, | 1.0 | | 5.00 |
| | Propyl Paraben, | | | |
| | Methyl Paraben | | | |
| Total | | 100 | | |

The formulation was prepared as described above for Example 1.

### Example 3

The therapeutic formulations described herein are analyzed by applying to porcine skin in a topical dose study. One male pig and one female pig are employed in the study, with each animal having 6 test areas. For each pig, three test areas are dosed once per day for seven days, while the remaining three areas are dosed only one time on day one. All animals should receive the same dose of the formulation, which is approximately 200 mg applied to a 121 cm² test area.

After application, skin samples are obtained and analyzed by washing the skin with a mild soap and water mixture. A full thickness skin section is removed by scalpel, gently rinsed with deionized water, and individual skin layers are separated. The layers are individually extracted with an organic solvent (i.e., isopropanol) and the amount of active ingredient (i.e., ibuprofen) is quantified by HPLC.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An aqueous cream for topical application to the skin comprising about 5% (w/w) to about 15% (w/w) diethylene glycol monoethyl ether, about 4% (w/w) to about 8% (w/w) tropical or vegetable oil, about 3% (w/w) to about 6.5% (w/w) ibuprofen, and about 56% (w/w) to about 68% (w/w) water, wherein said cream is stable at a temperature of 40° C. for a period of at least one month.

2. The aqueous cream of claim 1, comprising about 8% (w/w) to about 12% (w/w) diethylene glycol monoethyl ether, about 5% (w/w) to about 7% (w/w) tropical or vegetable oil, about 4% (w/w) to about 5.5% (w/w) ibuprofen, and about 60% (w/w) to about 64% (w/w) water, wherein said cream is stable at a temperature of 40° C. for a period of at least one month.

3. A aqueous cream for topical application to the skin comprising greater than about 5% by weight diethylene glycol monoethyl ether, tropical or vegetable oil, an excipient component, ibuprofen, and water, wherein said cream is stable at temperatures of at least 40° C. for a period of at least one month.

4. The aqueous cream of claim 3, comprising greater than about 3% (w/w) of tropical or vegetable oil.

5. The aqueous cream of claim 3, comprising greater than about 3% (w/w) of ibuprofen.

6. The aqueous cream of claim 3, comprising greater than about 55% (w/w) of water.

7. The aqueous cream of claim 3, comprising greater than about 10% (w/w) of excipient component.

8. The aqueous cream of claim 3, comprising less than about 10% (w/w) of tropical or vegetable oil.

9. The aqueous cream of claim 3, comprising less than about 10% (w/w) of ibuprofen.

10. The aqueous cream of claim 3, comprising less than about 6% (w/w) of ibuprofen.

11. The aqueous cream of claim 3, comprising less than about 75% (w/w) of water.

12. The aqueous cream of claim 3, comprising less than about 25% (w/w) of excipient component.

13. The aqueous cream of claim 3, comprising about 0.2% (w/w) to about 12% (w/w), about 0.5% (w/w) to about 11% (w/w), about 1% (w/w) to about 10% (w/w), about 2% (w/w) to about 9% (w/w), about 4% (w/w) to about 8% (w/w), about 5% (w/w) to about 7% (w/w), or about 6% (w/w) of tropical or vegetable oil.

14. The aqueous cream of claim 3, comprising about 0.2% (w/w) to about 10.5% (w/w), about 0.6% (w/w) to about 9.5% (w/w), about 0.8% (w/w) to about 8.5% (w/w), about 1.5% (w/w) to about 7.5% (w/w), about 3% (w/w) to about 6.5% (w/w), about 4% (w/w) to about 5.5% (w/w), or about 5% (w/w) of ibuprofen.

15. The aqueous cream of claim 3, wherein the molar ratio of ibuprofen to coconut oil is in the range from about 10:1 to 1:10, about 5:1 to 1:5, about 2:1 to 1:2, or about 1:1.2.

16. The aqueous cream of claim 1, wherein the molar ratio of ibuprofen to diethylene glycol monoethyl ether is in the range from about 10:1 to 1:10, about 8:1 to 1:8, about 4:1 to 1:4, or about 1:2.

17. The aqueous cream of claim 3, wherein the molar ratio of water to ibuprofen is in the range from about 100:1 to 1:10, about 50:1 to 1:5, about 40:1 to 1:4, about 30:1 to 1:3, about 25:1 to 1:2, about 20:1 to 1:1, or about 12.5:1.

18. An aqueous cream for topical application to the skin comprising tropical or vegetable oil, an excipient component, ibuprofen, water, and diethylene glycol monoethyl ether, wherein said cream is stable at a temperature of 40° C. for a period of at least one month.

19. An aqueous cream for topical application to the skin comprising tropical or vegetable oil, an excipient component, ibuprofen, water, and means for enhancing topical penetration, wherein said cream is stable at a temperature of 40° C. for a period of at least one month.

20. The cream of claim 3, wherein the tropical or vegetable oil is coconut oil.

21. A therapeutic cream for application to skin comprising coconut oil, a trigylceride, glycerol, xanthan gum, PEG-6 stearate, ethylene glycol palmitostearate, PEG-32 stearate, diethylene glycol monoethyl ether, phenoxyethanol, propyl paraben, methyl paraben, ibuprofen, and water, wherein said cream is stable at a temperature of 40° C. for a period of at least one month.

22. A method of reducing pain comprising applying to skin a composition of claim 3.

23. The method of claim 22, wherein the pain is from inflammation.

24. The method of claim 22, wherein the pain is of one or more joints.

25. The method of claim 24, wherein the pain of one or more joints is from arthritis.

26. The method of claim 22, comprising greater than about 4% (w/w) of tropical or vegetable oil.

27. The method of claim 22, comprising greater than about 3% (w/w) of ibuprofen.

28. The method of claim 22, comprising greater than about 55% (w/w) of water.

29. The method of claim 22, comprising greater than about 10% (w/w) of excipient component.

30. The method of claim 22, comprising less than about 10% (w/w) of tropical or vegetable oil.

31. The method of claim 22, comprising less than about 10% (w/w) of ibuprofen.

32. The method of claim 22, comprising less than about 6% (w/w) of ibuprofen.

33. The method of claim 22, comprising less than about 70% (w/w) of water.

34. The method of claim 22, comprising less than about 20% (w/w) of excipient components.

35. The method of claim 22, comprising about 0.2% (w/w) to about 12% (w/w), about 0.5% (w/w) to about 11% (w/w), about 1% (w/w) to about 10% (w/w), about 2% (w/w) to about 9% (w/w), about 4% (w/w) to about 8% (w/w), about 5% (w/w) to about 7% (w/w), or about 6% (w/w) of tropical or vegetable oil.

36. The method of claim 22, comprising about 0.2% (w/w) to about 10.5% (w/w), about 0.6% (w/w) to about 9.5% (w/w), about 0.8% (w/w) to about 8.5% (w/w), about 1.5% (w/w) to about 7.5% (w/w), about 3% (w/w) to about 6.5% (w/w), about 4% (w/w) to about 5.5% (w/w), or about 5% (w/w) of ibuprofen.

37. The method of claim 22, comprising about 35% (w/w) to about 90% (w/w), about 40% (w/w) to about 85% (w/w), about 45% (w/w) to about 80% (w/w), about 50% (w/w) to about 75% (w/w), about 55% (w/w) to about 70% (w/w), about 60% (w/w) to about 65% (w/w), or about 62% (w/w) of water.

38. The method of claim 22, comprising about 5% (w/w) to about 20% (w/w), about 6% (w/w) to about 19% (w/w), about 7% (w/w) to about 18% (w/w), about 8% (w/w) to about 17% (w/w), about 9% (w/w) to about 16% (w/w), about 10% (w/w) to about 15% (w/w), or about 13% (w/w) of excipient component.

39. The method of claim 22, wherein the molar ratio of ibuprofen to coconut oil is in the range from about 10:1 to 1:10, about 5:1 to 1:5, about 2:1 to 1:2, or about 1:1.2.

40. The method of claim 22, wherein the molar ratio of ibuprofen to diethylene glycol monoethyl ether is in the range from about 10:1 to 1:10, about 8:1 to 1:8, about 4:1 to 1:4, or about 1:2.

41. The method of claim 22, wherein the molar ratio of water to ibuprofen is in the range from about 100:1 to 1:10, about 50:1 to 1:5, about 40:1 to 1:4, about 30:1 to 1:3, about 20:1 to 1:2, about 15:1 to 1:1, or about 12.5:1.
